(19) United States
(12) Patent Application Publication
(10) Pub. No.: US 2005/0287091 A1
(43) Pub. Date: Dec. 29, 2005

(54) Color compositions and methods of using the same

(75) Inventor: Richard Kaiser, Allentown, PA (US)

Correspondence Address: AKERMAN SENTERFITT, P.O. BOX 3188, WEST PALM BEACH, FL, 33402-3188, US

(73) Assignees: Spectrum Associates LLC, Boca Raton, FL (US); Longoria Design LLC, Miami, FL (US); Kennedy/Matsumoto Design LLC, Lantana, FL (US)

(21) Appl. No.: 10/877,611

(22) Filed: Jun. 25, 2004

(51) Int. Cl. A61K 7/021; A61K 7/13

(52) U.S. Cl. 424/63

(57) ABSTRACT

A color composition and a method of using this color composition. The color composition may be used to color various portions of the body, such as hair, lips, face, and the like. The color composition results in a semi-solid material that may by applied using pressure. As such, the color composition may be easily applied. Various embodiments of the color composition result in differing colors and metallic colors. The color compositions may be used such that the same color results even though the color of the underlying substrate is substantially different.

### FIELD OF THE INVENTION

This invention is directed generally to color compositions, and more particularly to color compositions and methods for applying these color compositions to different portions of an individual's anatomy.

### BACKGROUND OF THE INVENTION

Many individuals color one or more portions of the anatomy for a variety of different reasons. For example, many individuals may color certain portions of their skin, such as their lips, face and hands. Applying color to the lips and/or face is often done by many women as part of their daily routine. However, on various occasions, men may use color as well, such as for Halloween or costume parties. In many instances, these individuals may wish to try the coloring for a shorter period of time, with the ability to remove the coloring if they do not like their new look and/or once the special occasion is finished.

For the same or similar reasons, some individuals color their hair. Hair coloring may be undertaken to change or cover the color of hair for many different reasons. For instance, hair is often colored to cover hair that has turned gray, to lighten or change the shade of hair, to highlight or lowlight hair, to try something new, or for a special occasion, again, such as dressing up for Halloween. The hair coloring procedures used to generate these results are often markedly different and use different coloring materials. The hair coloring materials may be dyes that may or may not be easily washed from the colored hair with conventional shampoo and water, or other materials. Again, in many instances, these individuals may wish to try the coloring for a shorter period of time, with the ability to remove the coloring if they do not like their new look or once the special occasion is finished.

The application of cosmetic and personal care products to the skin almost always involves the formation of a film on the skin. For example, application of a foundation to the face involves formation of a colored film on the skin. The same is true of products such as lipstick, eye shadow, blush, and nail enamel. The product is applied to the appropriate keratinous surface and allowed to dry. A film is formed that adheres to the skin for the appropriate period of time before being removed by chemical processes, or washed off with soap and/or water.

Accordingly, film forming polymers are widely used in the cosmetic industry. The ability to form films on skin, lips, or hair is one of the main means of providing a cosmetic benefit. For example, materials such as film forming cationic polymers are found in hair conditioning agents. They are capable of depositing on the hair to form a film or coating which provides benefits varying from shine, to softness, manageability, and similar characteristics. Similarly, various film forming polymers may be found in color cosmetics such as lipsticks. These polymers may affect lipstick wear and adhesion, in addition to serving as a film to hold the pigments in place on the lips. Also, nail enamel compositions usually contain, as the main component, some type of film forming polymer. Such compositions are typically wholly liquid or comprise liquids which contain solid particulates in suspension. They are applied as liquids and, after drying, the film formed on the nail usually lasts for at least several days, although a key feature for such cosmetic applications is their removability. It is thus beneficial to provide compositions which are “semi-permanent,” meaning that they demonstrate high durability on the coated substrate, but yet may be easily removed at will by the user using mechanical methods such as wiping or absorbing into a porous medium such as a tissue, as with lipsticks; or by chemical methods, for example, the removal of a nail enamel using an organic solvent such as ethyl acetate or acetone. In general, the types of polymeric materials used in cosmetics have a significant impact on providing compositions with improved properties. Thus, cosmetics companies are always searching for new and different polymers to provide properties which improve cosmetic performance.

Most film forming cosmetic and personal care products contain a polymeric material as the primary film former. The term “film former” as used herein refers to a material that, upon drying, produces a continuous film on keratinous substrates such as skin, hair, or nails. The term “film forming polymer” as used herein refers to a film former that is in the polymeric form. A variety of polymers have film forming properties: they may be natural polymers, synthetic polymers, or polymers that have both natural and synthetic portions. While the polymers available today have a myriad of properties, most cosmetics and personal care products contain other ingredients to further modify the properties of the composition and improve aesthetics.

One of these additional ingredients is a colorant. Many make-up compositions, such as free or compact powders, foundations, face powders, eye shadows, lipsticks, concealers, blushers, mascaras, eyeliners, lip pencils or eye pencils or make-up products for the body, include a suitable vehicle and coloring agents of various natures, intended to give these compositions a certain color, before and/or after applying them to the skin, the lips and/or hair.

These coloring agents may be lakes, inorganic or organic pigments and/or pearlescent pigments, or, alternatively, dyes. However, many of these agents may present problems. Inorganic pigments have the advantage of being relatively stable, but have the drawback of giving rather dull, pale colors. Organic lakes have the advantage of giving the compositions lively colors, but are mostly unstable with respect to light, temperature or pH. As for pearlescent pigments, they allow varied, but never intense, colors to be obtained, with iridescent effects, but these are usually fairly weak.

Accordingly, it would be beneficial to provide a color composition that may be used to color various portions of a person's anatomy, such as hair, lips, face, hands, or the like. It would also be beneficial to provide a color composition that is semi-permanent such that it may be applied and removed after a short period of time. It would also be beneficial to provide a color composition that provides a more visible color, regardless of the color of the substrate to which it was applied.

### SUMMARY OF THE INVENTION

The present invention provides a color composition and a method of applying this composition to color various portions of the body, such as hair, lips, face, and the like. The color composition results in a semi-solid material that may by applied using slight pressure. As such, the color composition may be easily applied. Various embodiments of the color composition result in differing colors and may even be used for metallic colors. In some embodiments, the color compositions may be used such that the same color results even though the color of the underlying substrate is substantially different, such as blonde hair versus brunette hair.

In particular, in one aspect, the present invention provides a color composition including from about 20 to about 35% by weight of water, from about 20 to about 35% by weight of a co-solvent, from about 10 to about 30% by weight of sodium stearate, and from about 15 to about 30% by weight of a coloring agent. In another embodiment, the color composition includes from about 1 to about 5% by weight of a silicone polymer. In another embodiment, the color composition includes from about 27 to about 32% by weight of water, from about 26 to about 31% by weight of the co-solvent, and from about 12 to about 15% by weight of sodium stearate. In yet another embodiment, the color composition includes from about 20 to about 25% by weight of the coloring agent. In still another embodiment, the color composition includes an additional component selected from a dispersant, bactericide, fungicide, defoaming agent, and a combination thereof. In yet another embodiment, the co-solvent is propylene glycol. In still another embodiment, the coloring agent includes titanium dioxide and a colorant. In yet another embodiment, the coloring agent includes from about 5 to about 20% of titanium dioxide and about 5 to about 20% of the colorant. In still another embodiment, the color composition includes from about 7 to about 15% of titanium dioxide and about 7 to about 15% of the colorant. In yet another embodiment, the color composition includes from about 15 to about 30% of a metallic coloring agent. In still another embodiment, the color composition includes about 20% of the metallic coloring agent.

In another aspect, the present invention provides a color composition including from about 27 to about 32% by weight of water, from about 26 to about 31% by weight of a co-solvent, from about 12 to about 15% by weight of sodium stearate, and from about 20 to about 25% by weight of a coloring agent. In another embodiment, the color composition includes from about 1 to about 5% by weight of a silicone polymer. In another embodiment, the co-solvent is propylene glycol. In still another embodiment, the coloring agent includes titanium dioxide and a colorant. In yet another embodiment, the color composition includes from about 7 to about 15% of titanium dioxide and about 7 to about 15% of the colorant. In yet another embodiment, the color composition includes about 20% of a metallic coloring agent. In still another embodiment, the color composition includes an additional component selected from a dispersant, bactericide, fungicide, defoaming agent, and a combination thereof.

In yet another aspect, the present invention provides a color composition including water, a co-solvent, sodium stearate, a coloring agent, and a silicone polymer. In another embodiment, the co-solvent is propylene glycol. In still another embodiment, the coloring agent includes titanium dioxide and a colorant. In yet another embodiment, the color composition includes an additional component selected from a dispersant, bactericide, fungicide, defoaming agent, and a combination thereof.

In still another aspect, the present invention provides a method of using a color composition including the steps of applying the color composition to a surface, wherein the color composition includes water, a co-solvent, sodium stearate, a coloring agent, and a silicone polymer. In another embodiment, the surface is selected from skin, lips, hair, and a combination thereof. In still another embodiment, an applicator is used to apply the color composition. In yet another embodiment, the co-solvent is propylene glycol. In still another embodiment, the coloring agent includes titanium dioxide and a colorant.

In yet another aspect, the present invention provides a color composition including from about 20 to about 35% by weight of water, from about 20 to about 35% by weight of a co-solvent, from about 10 to about 30% by weight of a liquid-crystal forming polymer, from about 15 to about 30% by weight of a coloring agent, and from about 1 to about 5% by weight of a silicone polymer. In another embodiment, the color composition includes from about 27 to about 32% by weight of water, from about 26 to about 31% by weight of the co-solvent, from about 12 to about 15% by weight of the liquid-crystal forming polymer, and from about 20 to about 25% by weight of the coloring agent. In still another embodiment, the color composition includes an additional component selected from a dispersant, bactericide, fungicide, defoaming agent, and a combination thereof. In yet another embodiment, the co-solvent is propylene glycol. In still another embodiment, the liquid-crystal forming polymer is sodium stearate. In still another embodiment, the coloring agent includes titanium dioxide and a colorant. In yet another embodiment, the coloring agent includes from about 5 to about 20% of titanium dioxide and about 5 to about 20% of the colorant. In still another embodiment, the color composition includes from about 7 to about 15% of titanium dioxide and about 7 to about 15% of the colorant. In yet another embodiment, the color composition includes from about 15 to about 30% of a metallic coloring agent. In still another embodiment, the color composition includes about 20% of the metallic coloring agent.

These and other uses will become apparent upon review of the detailed description below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a depiction of the formation of liquid crystals used in a base according to one embodiment of the present invention.

FIG. 2 is a diagram demonstrating possible mixing ratios of three components according to one embodiment of the present invention.

### DETAILED DESCRIPTION OF THE INVENTION

The present invention is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The color composition of the present invention is contemplated to be used on one or more parts of an individual's anatomy, such as by using as a cosmetic. As such, it is beneficial for the composition to include materials that are safe, nontoxic, and/or capable of being approved as cosmetic grade. In addition, as the color compositions may be applied for cosmetic purposes, it may be beneficial for the compositions to suspend color, dry quickly, stick to the substrate, and/or wash off with the application of soap or another cleaning agent. Also, the firmness of the compositions may be varied, depending on the selected characteristics that may be beneficial for application to the particular body part.

Accordingly, in one aspect of the present invention, the color composition utilizes liquid crystal technology as compositions made using this technology are safe, are suitable for application to skin and hair, and/or may be made into a wide range of hardness. The liquid crystal technology is used to produce the base to the color composition. The base, in conjunction with a coloring agent and optional ingredients, forms the color composition of the present invention.

Accordingly, the base of the color compositions of the present invention, in one embodiment, use sodium stearate. Sodium stearate has a structure wherein one end of the molecule is hydrophilic (water loving) and the other end is oliophilic (oil-loving). As such, when sodium stearate is dissolved in water and a miscible solvent, the sodium stearate molecule is capable of orienting one end in each solvent. Due to the optimum packing order of the sodium stearate molecules, tubes may be formed. These tubes can actually partition the water and the co-solvent. While the present embodiment is directed to the use of sodium stearate, it is contemplated that other liquid-crystal forming polymers may also be used in the present invention besides sodium stearate. Any polymer capable of forming a liquid-crystal may be used including, but not limited to, other stearate polymers.

In addition to sodium stearate and water, the compositions of the present invention use a co-solvent. The co-solvent may be any co-solvent capable of forming a color composition with sodium stearate and water. In one embodiment, the co-solvent is propylene glycol, although other glycols may also be used, such as ethylene glycol, butylene glycol, as well as others. When sodium stearate is dissolved in water and propylene glycol, a liquid crystal forms, with the oliophilic end of the sodium stearate oriented toward the propylene glycol, and water oriented at the hydrophilic end of the sodium stearate. As the material cools, the crystals form, with the length of the crystal being determined by the rate of cooling. The faster the cooling rate, the shorter the crystal and the shorter the cooling rate, the longer the crystal.

The length of the crystal has an effect on the mechanical properties of the resulting composition. Longer crystals are generally more flexible and, as a result, are generally weaker than shorter crystals. As such, shorter crystals are stronger. For example, a composition having a mixture of 33.3% short crystals and 66.7% liquid may be as strong as a composition that is 100% hard wax.

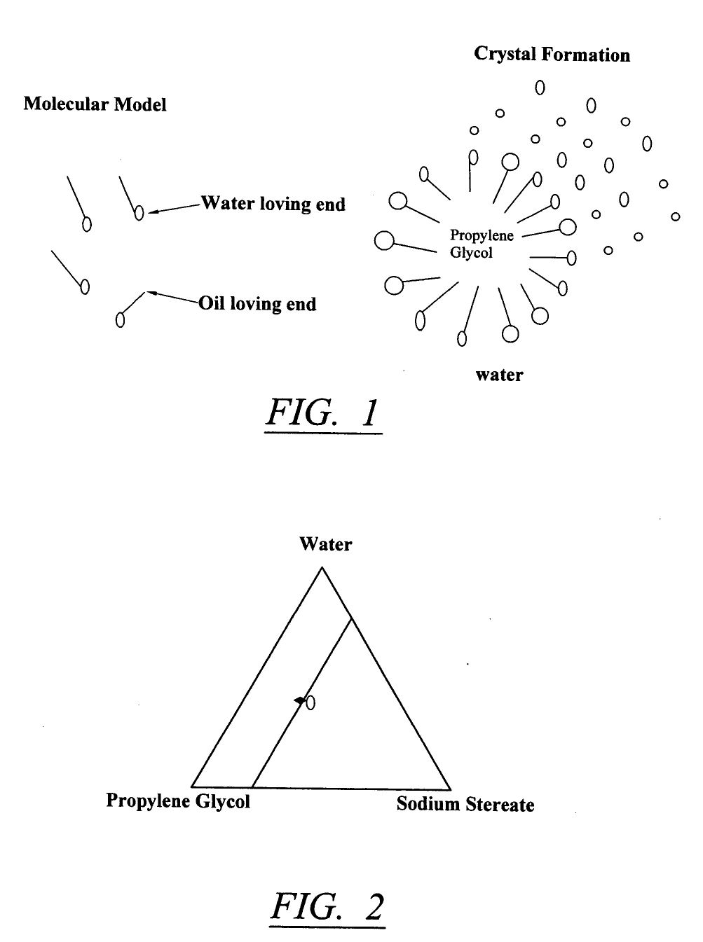

FIG. 1 provides a depiction of the formation of liquid crystals in an embodiment wherein the co-solvent is propylene glycol and the liquid-crystal forming polymer is sodium stearate. As seen, the sodium stearate has a hydrophilic end (as shown with a water molecule attached thereto) and an oliophilic end. Crystals form when the oliophilic ends of the sodium stearate align towards the propylene glycol.

The overall physical properties of the color compositions of the present invention may be determined by the ratio of the liquid-crystal forming polymer, water, and co-solvent. In an embodiment wherein sodium stearate is the liquid-crystal forming polymer, the ratio of the water to co-solvent to sodium stearate determines the concentration of the crystals and the continuous phase. FIG. 2 provides a diagram demonstrating the possible mixing ratios of these three components in an embodiment wherein the co-solvent is propylene glycol.

As shown in FIG. 2, the dot in the center represents the center of the diagram wherein each component is present in equal amounts. Formulations having more water and/or sodium stearate tended to be too hard or too mushy. Formulations having an excess amount of propylene glycol in relation to the water also tended to be too mushy. It was discovered that compositions in the area represented by the diamond offered the most beneficial characteristics, although formulations around the diamond may also be used, depending on the selected characteristics of the final color composition and/or the co-solvent used. Nevertheless, in those embodiments represented by the diamond, the water and propylene glycol are present in substantially equal amounts, wherein the sodium stearate is present in a lesser amount. In select embodiments, the amount of sodium stearate in the color composition is from about 15 to about 50% less than the amount of the water and the co-solvent. As such, in one embodiment, the present invention includes from about 20 to about 35% by weight of the total color composition of water and the co-solvent, and from about 10 to about 30% by weight of sodium stearate. In another embodiment, the present invention includes from about 26 to about 32% by weight of the total color composition of water and the co-solvent, and from about 12 to about 15% by weight of sodium stearate.

The base of the present invention may also include a component to improve the spreadability of the color composition, thereby making the composition easier to apply to the selected substrate. In one embodiment, this component is a silicone polymer. One example of a silicone polymer that may be used is Gafquat H—Si, which is manufactured by International Specialty Products (ISP, Wayne, N.J.). The amount of the silicone polymer added may be, in one embodiment, from about 1 to about 5% by weight of the total color composition. In another embodiment, the amount of silicone polymer is about 2% by weight of the total color composition.

Once the base has been formed, a coloring agent is then added. One of the problems with coloring agents is that not all colors appear the same when applied to substrates having different colors. For example, a blue color applied to hair may appear different than the same blue color applied to black hair as the “blueness” is much less noticeable on the darker substrate. Accordingly, in select embodiments of the present invention, the coloring agent used results in a color composition that appears substantially the same, regardless of the color of the substrate to which it is applied, such as hair, skin, and/or lips.

In one embodiment, the coloring agent includes titanium dioxide and a colorant. The titanium dioxide is used to effectively “hide” or cover up the color of the underlying substrate such that the color of the colorant may be seen regardless of the color of the underlying substrate. As such, the color composition may be used on all types and colors of hair, lips and/or skin. In one embodiment, the amount of titanium dioxide is from about 5 to about 20% by weight of the total color composition. In another embodiment, the amount of titanium dioxide is from about 7 to about 15% by weight of the total color composition. In alternative embodiments, other materials besides titanium dioxide may be used including, but not limited to, barium sulfate, aluminum silicates, calcium carbonates, and combinations thereof.

When titanium dioxide or a related material is used as part of the coloring agent, the coloring agent also includes a colorant. The colorant is the component that actually provides the color that is seen. The amount of colorant used may vary depending on the amount of titanium dioxide, the color of the colorant, and/or the selected degree of color for the finished color composition. In general, in one embodiment, the total amount of titanium dioxide and colorant is from about 15 to about 30% by weight of the total color composition. In another embodiment, the total amount of titanium dioxide and colorant is from about 20 to about 25% by weight of the total color composition. Accordingly, in one embodiment, the amount of colorant is from about 5 to about 20% by weight of the total color composition. In another embodiment, the amount of colorant is from about 7 to about 15% by weight of the total color composition. Examples of colorants useful in the present invention include, but are not limited to, Sandsperse Blue WF253, Napthol Red 2R Dispersion, and Optiflow Yellow 74. Other colorants that may be used include those listed by the Food and Drug Administration under Color Additives Approved for Use in Cosmetics (Part 73, Subpart C and Part 74, Subpart C) as currently listed and/or as amended in the future.

In another embodiment, the color composition produces a metallic color through the use of a metallic coloring agent. In those embodiments using a metallic coloring agent, no titanium dioxide is used. The metallic coloring agent may be, in one embodiment, a mica-based metallic. As no titanium dioxide is necessary, additional amounts of the metallic coloring agent may be used in the final color composition. Accordingly, in one embodiment, the amount of the metallic coloring agent is from about 15 to about 25% by weight of the total color composition. In another embodiment, the amount of the metallic coloring agent is about 20% by weight of the total color composition. Examples of metallic coloring agents useful in the present invention include, but are not limited to, Dyna Color 98392B15A BlueGreen, Allair Type 303 Gold, and Dyna Color RB639XV19A. Other metallic coloring agents that may be used include those metallic coloring agents listed by the Food and Drug Administration under Color Additives Approved for Use in Cosmetics (Part 73, Subpart C and Part 74, Subpart C) as currently listed and/or as amended in the future.

In addition to the base and the coloring agent, the color compositions of the present invention may include optional components that may be included for a variety of purposes. For example, in one embodiment, the color composition includes a dispersant for preventing agglomeration between the pigment particles in the color composition. One example of a dispersant that may be used in the present invention is Tamol 731, a sodium salt of a carboxylated polyelectrolyte available from Rohm & Haas (Philadelphia, Pa.). The dispersant may be added in an amount of from about 0 to about 5% by weight of the total color composition. Any other dispersant may be used, including those manufactured by Olin, Rohm & Haas, Stefan, as well as other dispersant manufacturers. Examples of dispersants include, without limitation, soluble salts of low molecular weight homopolymers or copolymers of polyacrylic acids, partially hydrolyzed polyacrylamides, maleic anhydride copolymers, and polyaspartic acid.

In another embodiment, the color composition includes a bactericide to prevent growth of bacteria in the product prior to use. One example of a bactericide that may be used in the present invention is Germall®, a member of the substituted imidazolidinyl urea family of compounds available from ISP. The bactericide may be added in an amount of from about 0 to about 1% by weight of the total color composition. In addition, other bactericides and fungicides may also be used.

In another embodiment, the color composition includes a defoaming agent to help control foaming during production of the compositions of the present invention. One example of a defoaming agent that may be used in the present invention is Foamstar™ A-12, a polymer available from Cognis (Cincinnati, Ohio). The defoaming agent may be added in an amount of from about 0 to about 3% by weight of the total color composition.

The color compositions of the present invention may be made by mixing the water and co-solvent together, then heating to a temperature above which the sodium stearate will melt, generally above about 60° C., to form the base. Then, the coloring agent and any optional compounds may be added and the mixture heated to a temperature of from about 90 to about 100° C. to thoroughly mix all of the components. Then, the mixture may be gradually cooled to form the liquid crystals and the color compositions. The rate of cooling may be controlled to control the final strength of the color composition, wherein a faster rate of cooling results in a softer composition and a slower rate of cooling results in a firmer composition.

The compositions of the present invention may be used to color a variety of different surfaces including, but not limited to, various parts of an individual's anatomy. These various anatomy parts of an individual may include, but are not limited to, the hair, skin, lips or other surface of an individual. The compositions may be applied in any known manner, such as forming the composition into a stick and applying to the hair, lips, and/or face. In another embodiment, the compositions may be formed into a compact or any other suitable shape and applied using an applicator. As used herein, an “applicator” is any item that may be used to apply a semi-solid or substantially solid material to a surface including, but not limited to, fingers, a brush, a pad, cotton, a sponge, a fabric, and the like.

The foregoing is provided for purposes of illustrating, explaining, and describing embodiments of this invention. Modifications and adaptations to these embodiments will be apparent to those skilled in the art and may be made without departing from the scope or spirit of this invention.

### EXAMPLES

### Example 1

An example of a blue composition made according to various aspects of the present invention. In a mixing vessel, the following components were added:

| | | |
|---|---|---|
| Deionized water | 28 | parts |
| Propylene Glycol | 27.8 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and 15 parts titanium dioxide were added. The composition was mixed until all lumps were dispersed and the fluid appeared to be free of grit. Then, the colorant was added, 7 parts of Sandsperse Blue WF253 and the composition was heated until 80° C. Then 15 parts sodium stearate was added while ensuring the temperature did not drop below 60° C. The vessel was heated to 101° C. and held for 5 minutes before slowly cooling to form the final composition.

### Example 2

An example of a red composition made according to various aspects of the present invention. In a mixing vessel, the following components were added:

| | | |
|---|---|---|
| Deionized water | 28.8 | parts |
| Propylene Glycol | 28 | parts |
| Tamol 731 | 4 | parts |
| Gafquat H—Si | 2 | parts |
| Germal | 0.2 | parts |

The mixing vessel was heated to 80° C. and 7 parts titanium dioxide were added. The composition was mixed until all lumps were dispersed and the fluid appeared to be free of grit. Then, the colorant was added, 15 parts of Napthol Red 2R Dispersion and the composition was heated until 80° C. Then 15 parts sodium stearate was added while ensuring the temperature did not drop below 60° C. The vessel was heated to 101° C. and held for 5 minutes before slowly cooling to form the final composition.

### Example 3

An example of a yellow composition made according to various aspects of the present invention. In a mixing vessel, the following components were added:

| | | |
|---|---|---|
| Deionized water | 27.5 | parts |
| Propylene Glycol | 26.3 | parts |
| Tamol 731 | 4 | parts |
| Gafquat H—Si | 2 | parts |
| Germal | 0.2 | parts |

The mixing vessel was heated to 80° C. and 15 parts titanium dioxide were added. The composition was mixed until all lumps were dispersed and the fluid appeared to be free of grit. Then, the colorant was added, 10 parts of Optiflo Yellow and the composition was heated until 80° C. Then 15 parts sodium stearate was added while ensuring the temperature did not drop below 60° C. The vessel was heated to 101° C. and held for 5 minutes before slowly cooling to form the final composition.

### Example 4

An example of an electric blue metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Dyna Color 98392B15A BlueGreen, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Example 5

An example of a gold metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Allair Type 303 Gold, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Example 6

An example of a raspberry metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Dyna Color RB639XV19A, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Examples 7-14

Using the process set forth in Examples 1-6, eight additional formulations were prepared. The listing of the ingredients for these formulations is set forth in Table 1.

TABLE 1
HAIR PAINT INGREDIENTS AND FORMULATION

| CTFA Name | INCI | % | Supplier |
|---|---|---|---|
| Water | Aqua | 27.5-31.5 | Ten Li Distilled (local factory) |
| Propylene Glycol | Propylene Glycol | 26-30.5 | Lyondell Asia Pacific Ltd. |
| Sodium Stearate | Sodium Stearate | 12-13 | Stabilchem(M) Sdn Bhd |
| Isobutylene/MA Copolymer | Isobutylene/MA Copolymer | 4 | Rohm & Haas (TN Orctan) |
| Polyquaternium-28 | Polyquaternium-28 | 2 | International Special Products (TN Gavquat) |
| Methylparaben | Methylparaben | 0.03 | Nipa Laboratories Ltd. |
| Sodium Dehydroacetate | Sodium Dehydroacetate | 0.06 | Taisho Technos Co., Ltd. |
| May Contain: (Pigment) | | to 100 | |
| Mica & Titanium Dioxide | CI 77019 & CI 77891 | | Engelhard Corporation/Mearl |
| Titanium Dioxide | CI 77891 | | Sun Chemical |
| D&C Red No. 27 Al. Lake | CI 45410 | | Sun Chemical |
| FD&C Blue No. 1 Al. Lake | CI 42090 | | Sun Chemical or Wackhe [symbol] |
| Manganese Violet | CI 77742 | | Wackhe [symbol] |
| D&C Red No. 7 Cal. Lake | CI 15850 | | Wackhe [symbol] |
| Iron Oxides | CI 777491, CI77492, CI77499 | | Wackhe [symbol] |
| FD&C Yellow No. 5 Al. Lake | CI 19140 | | Wackhe [symbol] |

Notes:-
1. TN = Trade Name
2. % Variations due to different PH balance on different colours.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope of spirit of the disclosure. All such changes and modifications are intended to be included within the scope of the disclosure as defined by the appended claims.

1. A color composition, comprising:
from about 20 to about 35% by weight of water;
from about 20 to about 35% by weight of a co-solvent;
from about 10 to about 30% by weight of sodium stearate; and
from about 15 to about 30% by weight of a coloring agent.

2. The color composition of claim 1, wherein the composition further comprises:
from about 1 to about 5% by weight of a silicone polymer.

3. The color composition of claim 1, wherein the composition comprises:
from about 27 to about 32% by weight of water;
from about 26 to about 31% by weight of the co-solvent; and
from about 12 to about 15% by weight of sodium stearate.

4. The color composition of claim 1, wherein the composition comprises:
from about 20 to about 25% by weight of the coloring agent.

5. The color composition of claim 1, wherein the composition comprises an additional component selected from a dispersant, bactericide, fungicide, defoaming agent, and a combination thereof.

6. The color composition of claim 1, wherein is the co-solvent is a glycol.

7. The color composition of claim 1, wherein the coloring agent comprises titanium dioxide and a colorant.

8. The color composition of claim 7, wherein the coloring agent comprises from about 5 to about 20% of titanium dioxide and about 5 to about 20% of the colorant.

9. The color composition of claim 7, wherein the coloring agent comprises from about 8 to about 15% of titanium dioxide and about 7 to about 15% of the colorant.

10. The color composition of claim 1, wherein the coloring agent comprises from about 15 to about 30% of a metallic coloring agent.

11. The color composition of claim 10, wherein the coloring agent comprises about 20% of the metallic coloring agent.

12. A color composition, comprising:
from about 27 to about 32% by weight of water;
from about 12 to about 15% by weight of sodium stearate; and
from about 20 to about 25% by weight of a coloring agent.

13. The color composition of claim 12, wherein the composition further comprises:
from about 1 to about 5% by weight of a silicone polymer.

14. The color composition of claim 12, wherein is the co-solvent is a glycol.

15. The color composition of claim 12, wherein the coloring agent comprises titanium dioxide and a colorant.

16. The color composition of claim 15, wherein the coloring agent comprises from about 7 to about 15% of titanium dioxide and about 7 to about 15% of the colorant.

17. The color composition of claim 12, wherein the coloring agent comprises about 20% of a metallic coloring agent.

18. The color composition of claim 12, wherein the composition comprises an additional component selected from a dispersant, bactericide, fungicide, defoaming agent, and a combination thereof.

19-22. (canceled)

23. A method of using a color composition comprising:
applying the color composition to a surface;
wherein the color composition comprises water, a co-solvent, sodium stearate, a coloring agent, and a silicone polymer.

24. The method of claim 23, wherein the surface is selected from skin, lips, hair, and a combination thereof.

25. The method of claim 23, further comprising using an applicator to apply the color composition.

26. The method of claim 23, wherein is the co-solvent is a glycol.

27. The method of claim 23, wherein the coloring agent comprises titanium dioxide and a colorant.

28. A color composition, comprising:
from about 20 to about 35% by weight of water;
from about 20 to about 35% by weight of a co-solvent;
from about 10 to about 30% by weight of a liquid-crystal forming polymer;
from about 15 to about 30% by weight of a coloring agent; and
from about 1 to about 5% by weight of a silicone polymer.

29. The color composition of claim 28, wherein the composition comprises:
from about 27 to about 32% by weight of water;
from about 26 to about 31% by weight of the co-solvent;
from about 12 to about 15% by weight of liquid-crystal forming polymer; and
from about 20 to about 25% by weight of the coloring agent.

30. The color composition of claim 28, wherein the composition comprises an additional component selected from a dispersant, bactericide, fungicide, defoaming agent, and a combination thereof.

31. The color composition of claim 28, wherein is the co-solvent is a glycol.

32. The color composition of claim 28, wherein is the liquid-crystal forming polymer is sodium stearate.

33. The color composition of claim 28, wherein the coloring agent comprises titanium dioxide and a colorant.

34. The color composition of claim 33, wherein the coloring agent comprises from about 5 to about 20% of titanium dioxide and about 5 to about 20% of the colorant.

35. The color composition of claim 34, wherein the coloring agent comprises from about 8 to about 15% of titanium dioxide and about 7 to about 15% of the colorant.

36. The color composition of claim 28, wherein the coloring agent comprises from about 15 to about 30% of a metallic coloring agent.

37. The color composition of claim 36, wherein the coloring agent comprises about 20% of the metallic coloring agent.

38. A method for applying color to hair comprising the steps of:
applying to hair a composition comprising water, a co-solvent, sodium stearate, a coloring agent, and a silicone polymer;
wherein the composition adheres to the hair, but washes off with the application of a cleaning agent to the hair.

39. The method of claim 38, wherein the composition comprises:
from about 27 to about 32% by weight of water;
from about 26 to about 31% by weight of the co-solvent;
from about 12 to about 15% by weight of liquid-crystal forming polymer; and
from about 20 to about 25% by weight of the coloring agent.

40. The method of claim 39, wherein the co-solvent is a glycol.

41. The method of claim 40, wherein the glycol is propylene glycol.

42. The method of claim 38, wherein the cleaning agent is shampoo.

43. A method for temporarily changing the color of hair comprising the steps of:
applying a composition comprising water, a co-solvent, sodium stearate, a coloring agent, and a silicone polymer to hair; whereby the composition adheres to the hair to change an apparent color of the hair; and
washing the hair with a cleaning agent, whereby the hair is restored to its original color.

44. The method of claim 43, wherein the cleaning agent is shampoo.

45. A method for restoring an original color of hair after application of a color composition comprising water, a co-solvent, sodium stearate, a coloring agent, and a silicone polymer to hair comprising the steps of:
removing the composition from the hair by washing the hair with a cleaning agent.

46. The method of claim 45, wherein the cleaning agent is shampoo.

47. The color composition of claim 6, wherein the glycol is propylene glycol.

48. The color composition of claim 14, wherein the glycol is propylene glycol.

49. The method of claim 26, wherein the glycol is propylene glycol.

50. The color composition of claim 31, wherein the glycol is propylene glycol.
